(19) United States
(12) Patent Application Publication
(10) Pub. No.: US 2024/0358355 A1
(43) Pub. Date: Oct. 31, 2024

(54) System and Method for Monitoring a Vasculature for Sepsis

(71) Applicant: Bard Access Systems, Inc., Salt Lake City, UT (US)

(72) Inventors: Steffan Sowards, Salt Lake City, UT (US); William Robert McLaughlin, Bountiful, UT (US); Stefan Josef Fellner, Salt Lake City, UT (US)

(21) Appl. No.: 18/139,237

(22) Filed: Apr. 25, 2023

(51) Int. Cl. A61B 8/08; A61B 8/00

(52) U.S. Cl. CPC A61B 8/5223; A61B 8/0891; A61B 8/4472

(57) ABSTRACT

A system and method for monitoring sepsis in a patient. Logic processes ultrasound image data acquired by one or more pads applied to the patient. The logic determines vascular parameters values, such as blood vessel size or shape, and the logic further determines a sepsis status and/or progression from the vascular parameter values by performing an algorithm on the vascular parameter values. The algorithm is defined utilizing artificial intelligence techniques applied to an ongoing collection data sets acquired from a plurality of sepsis monitoring systems across a plurality of patients undergoing sepsis events, where each data set includes one or more vascular parameter values and a corresponding independently acquired sepsis status for the sepsis event. The data set may also include patient data such as weight, age, etc. The pads may be wirelessly coupled with a system module. The vascular parameters may include multiple vascular sites on the patient.

### BACKGROUND

Sepsis is the body's overwhelming and life-threatening response to infection that can lead to tissue damage, organ failure, and death. Like strokes or heart attacks, sepsis is a medical emergency that requires rapid diagnosis and treatment. Sepsis must be treated quickly and efficiently as soon as healthcare providers suspect it. If it is not recognized and treated quickly, sepsis can progress to severe sepsis and then to septic shock. It is reported that the chance of sepsis progressing to severe sepsis and septic shock, causing death, rises by 4% to 9% for every hour treatment is delayed. Septic shock is related to hypotension causing a decrease on blood flow to organs causing them to shut down.

Disclosed herein are systems and methods that monitor vascular parameters associated with sepsis, thereby providing early detection of sepsis and enabling rapid treatment.

### SUMMARY

Disclosed herein is a medical system that, according to some embodiments, includes a first pad applied to a skin surface of a patient, where the pad is configured to acquire an image of a first vasculature of the patient. The system further includes a system module having a console coupled with the first pad, where the console includes a processor and a memory having logic stored thereon that, when executed by the processor performs operations of the system. The operations may include (i) receiving the image of the first vasculature from the first pad, (ii) determining a value of a vascular parameter of the first vasculature from the image of the first vasculature, and (iii) determining a status of a sepsis of the patient based on the value of the vascular parameter of the first vasculature.

In some embodiments, the first pad includes a number of ultrasonic transducers extending across a patient contact surface of the first pad, the ultrasonic transducers configured to acquire the image of the first vasculature.

In some embodiments, the image of the first vasculature includes a first blood vessel, and the vascular parameter includes a size of the first blood vessel, a shape of the first blood vessel, or a blood flow rate through the first blood vessel.

In some embodiments, the vascular parameter is one of a plurality of vascular parameters that includes at least two of the size of the first blood vessel, the shape of the first blood vessel, or the blood flow rate through the first blood vessel.

In some embodiments, the operations further include (i) receiving a first image of the first vasculature from the first pad; (ii) receiving a second image of the first vasculature from the first pad, where the second image is received subsequent the first image; (iii) determining a first value of the vascular parameter of the first vasculature from the first image; (iv) determining a second value of the vascular parameter of the first vasculature from the second image; (v) determining a difference between the second value and the first value; and (vi) determining a progression of the sepsis based on the difference.

In some embodiments, the image of the first vasculature includes a second blood vessel of the first vasculature, and the vascular parameter includes at least one of a size of the second blood vessel, a shape of the second blood vessel, or a blood flow rate through the second blood vessel.

In some embodiments, the system further includes a second pad applied to the skin surface at a location separate from the first pad, where the second pad is configured to acquire an image of a second vasculature of the patient. In such an embodiment, the console is coupled with the second pad, and the operations further include (i) receiving the image of the second vasculature from the second pad, (ii) determining a value of a vascular parameter of the second vasculature from the image of the second vasculature, and (iii) determining the status of the sepsis based on the value of the vascular parameter of the second vasculature in combination with the value of the vascular parameter of the first vasculature.

In some embodiments, the determining a status of a sepsis includes performing an algorithm on the value of the vascular parameter, where the algorithm is defined at least partially by historical data from a plurality of sepsis events across a population of patients.

In some embodiments, the historical data include values of the vascular parameter and statuses of the sepsis that correspond to the values of the vascular parameter.

In some embodiments, the historical data further include corresponding patient data, and the patient data include one or more of weight, height, sex, age, race, or body mass index.

In some embodiments, the system module is communicatively coupled with an external computing device configured to (i) receive the value of the vascular parameter from the system module, (ii) generate the algorithm based on the value of the vascular parameter and the historical data, and (iii) transmit the algorithm to the system module.

In some embodiments, the operations further include comparing the status of the sepsis with a plurality of ranked sepsis statuses stored in the memory and providing a notification when the status of the sepsis exceeds a defined one of the ranked sepsis statuses.

Also disclosed herein is a method of determining a status of a sepsis of a patient that, according to some embodiments, includes collecting a plurality of data sets from a plurality of medical systems configured to determine a value of a vascular parameter of a patient undergoing a sepsis event, where each data set includes a recorded value of the vascular parameter and an independently acquired status of a sepsis corresponding to the recorded value of the vascular parameter. The method further includes (i) defining an algorithm that correlates the independent statuses of the sepsis with the recorded values of the vascular parameter across the plurality of data sets, (ii) operatively coupling a new patient undergoing a sepsis event with one of the medical systems (iii), determining a new value of the vascular parameter for the new patient, and (iv) performing the algorithm on the new value of the vascular parameter to determine a status of a sepsis of the new patient.

In some embodiments of the method, each medical system includes at least one pad having a number of ultrasonic transducers extending across a patient contact surface of the pad, the ultrasonic transducers configured to acquire the value of the vascular parameter.

In some embodiments of the method, the vascular parameter includes at least one of a size of a blood vessel, a shape of the blood vessel, or a blood flow rate through the blood vessel.

In some embodiments, the method further includes (i) determining a first new value of the vascular parameter; (ii) determining a second new value of the vascular parameter, where the second new value is acquired subsequent the first new value; (iii) determining a difference between the second new value and the first new value; and (iv) determining a progression of the sepsis based on the difference.

In some embodiments of the method, the determining a new value of the vascular parameter includes determining a new value of the vascular parameter for a first vasculature via a first pad and determining a new value of the vascular parameter for a second vasculature via a second, where the second vasculature is spaced away from the first vasculature.

In some embodiments of the method, each data set includes patient data that correspond to the vascular parameter and the status of the sepsis, and the patient data include one or more of weight, height, sex, age, race, or body mass index.

In some embodiments of the method, the one of the medical systems is communicatively coupled with an external computing device, and the method further includes (i) storing the plurality of data sets in a memory of the external computing device, (ii) transmitting new values of patient data for the new patient to the external computing device, (iii) defining the algorithm based on the plurality of data sets and the new values of patient data for the new patient, and (iv) receiving the algorithm from the external computing device.

In some embodiments, the method further includes (i) obtaining an independently acquired status of the sepsis of the new patient, (ii) transmitting the independent status to the external computing device, and (iii) storing the new value of the vascular parameter together with the independently acquired status in the data base as an additional data set.

These and other features of the concepts provided herein will become more apparent to those of skill in the art in view of the accompanying drawings and following description, which describe particular embodiments of such concepts in greater detail.

### DRAWINGS

FIG. 1 illustrates a sepsis monitoring system in use with a patient, in accordance with some embodiments.

FIGS. 2A-2C illustrate an ultrasound pad of the system of FIG. 1 acquiring various vascular parameters of a patient vasculature, in accordance with some embodiments.

FIG. 3 illustrates an exemplary table of data utilized by logic performing artificial intelligence techniques to define an algorithm that correlates sepsis status to vascular parameter values, in accordance with some embodiments.

FIG. 4 illustrates a block diagram of a system method of determining a status of a sepsis of a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. For example, the “vascular parameter” a used herein may one individual vascular parameter or a combination of plurality of individual vascular parameters.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

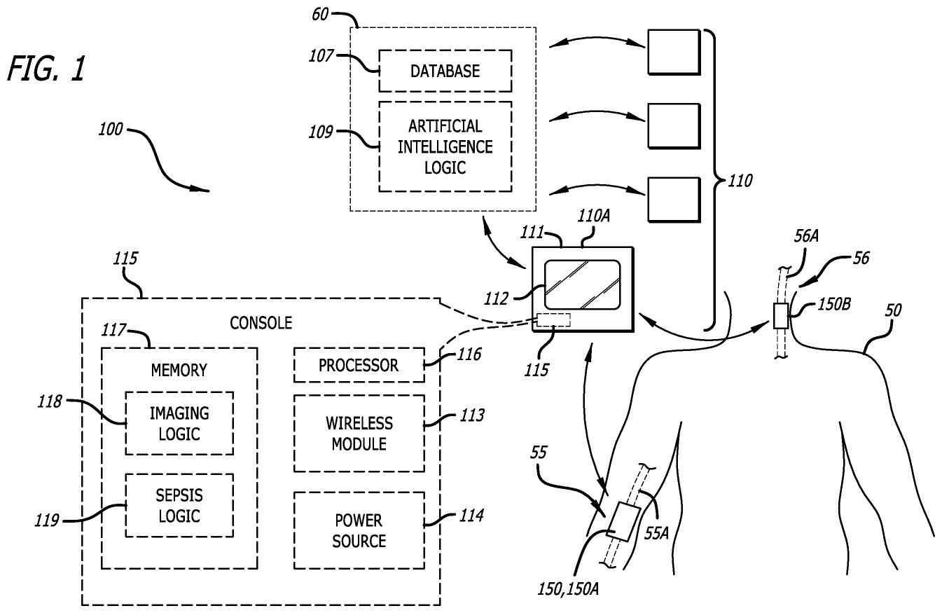

FIG. 1 illustrates a medical system 100 configured to determine a sepsis status, according to some embodiments. In some embodiments, the system 100 may detect an early sepsis condition and/or monitor the progression of the sepsis condition and provide a notification accordingly. The medical system 100 generally includes a plurality of sepsis monitoring systems 110 communicatively coupled with an external computing device 60. The external computing device 60 may be any suitable computing device, such as a network server, for example. In some embodiments, the external computing device 60 may be coupled with an electronic medical record system. An artificial intelligence (AI) logic 109 and a sepsis monitoring database 107 are stored in memory (e.g., a non-transitory computer-readable medium) of the external computing device 60. The AI logic 109 performs AI and/or Machine learning operations on the data stored in the sepsis monitoring database (database) 107 as further described below.

The sepsis monitoring system (system) 110A, which is one of the sepsis monitoring systems 110, is shown in use with a patient 50 undergoing a sepsis event. The system 110A is generally configured to detect, determine, and/or monitor the sepsis condition or status of the patient 50. The system 110A (as with each of the systems 110) includes a system module 111 and a number (e.g., 1, 2, 3 or more) pads 150, such as a first pad 150A and a second pad 150B, for example. The first pad 150A may be coupled (including acoustically coupled) with the patient 50 at a first target location 55 (including a first vasculature 55A) and the second pad 150B may be coupled (including acoustically coupled) with the patient 50 at a second target location 56 (including a second vasculature 56A). Additional pads 150 may be coupled with the patient 50 at additional target locations. In the description that the follows “the pad 150” refers to the first pad 150A and/or a second pad 150B. The pad 150 is configured to determine a value of a vascular parameter of a vasculature of the patient 50 adjacent (e.g., disposed beneath) the pad 150 as further described below. In some embodiments, one of the first vasculature 55A or the second vasculature 56A may be a central vasculature and the other one of the first vasculature 55A or the second vasculature 56A may be a peripheral vasculature.

The pad 150 may be attached to the patient in any suitable fashion. In some embodiments, the pad 150 may include a band or a cuff (not shown) configured to secure the pad 150 to the patient 50. In other embodiments, the pad 150 may include an adhesive patch (not shown) configured to secure the pad 150 to the patient 50.

The system 110A includes a console 115 and each of the pads 150 is communicatively coupled with the console 115. The pads 150 may be coupled with the console 115 via a wired or wireless connection. In the illustrated embodiment, the console 115 includes a processor (or multiple processors) 116 that executes logic stored in a memory 117 (e.g., a non-transitory computer-readable medium) such as the imaging logic 118 and the sepsis logic 119. A power source 114 of the console 115 may include an external source (i.e., a facility power source) or a battery. The console 115 may include a wireless module 113 to facilitate wireless communication between the pads 150 and the system module 11A and/or between the wireless the system module 110A and the external computing device 60. In some embodiments, system 110A may include (or be coupled with) a display 112 (e.g., a graphical user interface) configured to display notification information and/or receive input from a clinician.

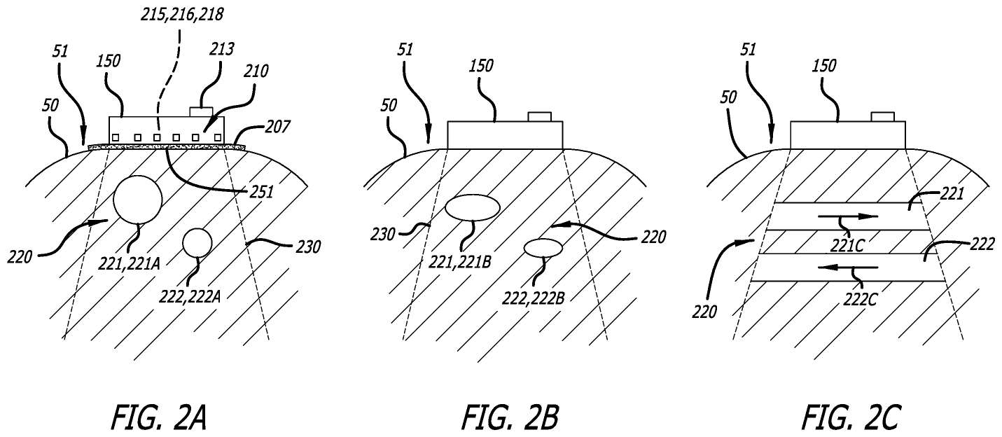

FIG. 2A illustrates a cross-sectional view of a portion of the patient 50 undergoing a sepsis event, where the cross-sectional view is perpendicular to a vasculature 220 of the patient 50. The pad 150 is applied to a skin surface 51 of the patient 50. In the illustrated embodiment, the pad 150 is configured to obtain an ultrasonic image 230 (which may be a live image) of the patient 50. The pad 150 may include plurality of ultrasonic transducers 210 (e.g., an array of ultrasonic transducers 210) disposed across patient contact surface 251 of the pad 150. The pad 150 includes a pad console 215 having a processor 216 executing image acquiring logic 218 that may include a portion of the imaging logic 118. In some embodiments as stated above, the pad console 215 may include a wireless module 213 to facilitate wireless communication with the system module 110A. The pad 150 (or more specifically the array of ultrasonic transducers 210) are configured to obtain the ultrasound image 230. In some embodiments, a gel 207 or other substance may be disposed between the pad 150 and the skin surface 51 to enhance the acoustic coupling of the pad 150 with the skin surface 51.

The imaging logic 118 is configured to identify anatomical elements within the ultrasound image 230. FIG. 2A shows a vasculature 220 including a first blood vessel 221 (e.g., a vein) and a second blood vessel 222 (e.g., an artery). As such, the imaging logic 118 is configured to identify the first blood vessel 221 and the second blood vessel 222. The imaging logic 118 is further configured to determine a vascular parameter or condition (or a number of individual vascular parameters) of the first blood vessel 221 and/or the second blood vessel 222, such as the first blood vessel size 221A and/or the second blood vessel size 222A. During a sepsis event, the blood pressure of the patient 50 may significantly decrease resulting in a decrease in the size of the blood vessel. As such, the size of a blood vessel may be indicative of a sepsis status. In some instances, the size (e.g., cross-sectional area) of a blood vessel, such as the first blood vessel 221 and/or the second blood vessel 222, for example, may change (i.e., decrease) during a sepsis event. The imaging logic 118 may be configured to determine/measure the first blood vessel size 221A and/or the second blood vessel size 222A. As such, the first blood vessel size 221A and/or the second blood vessel size 222A or a change in the first blood vessel size 221A and/or the second blood vessel size 222A may be a vascular parameter that is indicative of a sepsis status.

FIG. 2B illustrates a cross-sectional view of a portion of the patient 50 undergoing a sepsis event similar to FIG. 2A. In the embodiment depicted in FIG. 2B, the imaging logic 118 is configured to determine a shape of the first blood vessel 221 and/or the second blood vessel 222, such as the first blood vessel shape 221B and/or the second blood vessel shape 222B. As discussed above, during a sepsis event, the blood pressure of the patient 50 may significantly decrease which may cause a change in the first blood vessel shape 221B and/or the second blood vessel shape 222B. The imaging logic 118 may be configured to determine/measure the first blood vessel shape 221B and/or the second blood vessel shape 222B. For example, the first blood vessel shape 221B and/or the second blood vessel shape 222B may change from a round shape to an oval or flat shape. As such, the first blood vessel shape 221B and/or the second blood vessel shape 222B or a change in the first blood vessel shape 221B and/or the second blood vessel shape 222B may be a vascular parameter that is indicative of a sepsis status.

FIG. 2C illustrates a cross-sectional view of a portion of the patient 50 undergoing a sepsis event similar to FIG. 2A, where the cross-sectional view is a side view of the vasculature 220 of the patient 50. In the embodiment depicted in FIG. 2C, the imaging logic 118 is configured to determine a blood flow rate through the first blood vessel 221 and/or the second blood vessel 222, such as the first blood flow rate 221C and/or the second blood flow rate 222C. As discussed above, during a sepsis event, the blood pressure of the patient 50 may significantly decrease which may cause a change in first blood flow rate 221C and/or the second blood flow rate 222C. The imaging logic 118 may be configured to determine/measure a change (e.g., a decrease) in the first blood flow rate 221C and/or the second blood flow rate 222C. As such, a change (e.g., a decrease) in the first blood flow rate 221C and/or the second blood flow rate 222C may be a vascular parameter that is indicative of a sepsis status.

By way of summary, the size of one or more blood vessels, the shape of one or more blood vessels, or the blood flow rate through one or more blood vessels, as determined by one or more pads 150 may constitute a vascular parameter or condition (either individually or in combination) that is indicative of the sepsis status. The imaging logic 118 may receive and process the vascular parameter. The imaging logic 118 may store the vascular parameter (i.e., vascular parameter data) in the memory 117. The imaging logic 118 may also transmit the vascular parameter to the external computing device 60 for storage in the database 107 and/or processing by the AI logic 109. The sepsis logic 119 may determine the sepsis status based on the vascular parameter as further described below.

The AI logic 109 is generally configured to define a relationship (e.g., a correlation) between the vascular parameter and the sepsis status using AI techniques. In some embodiments, the AI techniques may include statistical processes, such as linear regression for example. The AI logic 109 receives data sets from the plurality of sepsis monitoring systems 110 and may define an algorithm based on the correlation between the vascular parameter and the sepsis status. The AI logic 109 may, in some embodiments, transmit the algorithm to the plurality of sepsis monitoring systems 110, such as the system 110A to enable the sepsis logic 119 to determine and/or monitor the sepsis status of the patient 50.

In some embodiments, imaging logic 118 may receive a first image of the first vasculature 55A from the first pad 150A. The imaging logic 118 may also receive a second image of the first vasculature 55A, where the second image is received subsequent the first image. The imaging logic 118 determine a first value of the vascular parameter of the first vasculature 55A from the first image and determine a second value of the vascular parameter of the first vasculature 55A from the second image. The imaging logic 118 may then determine a difference between the second value and first value. The sepsis logic 119 may then determine a progression of the sepsis status based on the difference between the second value and first value.

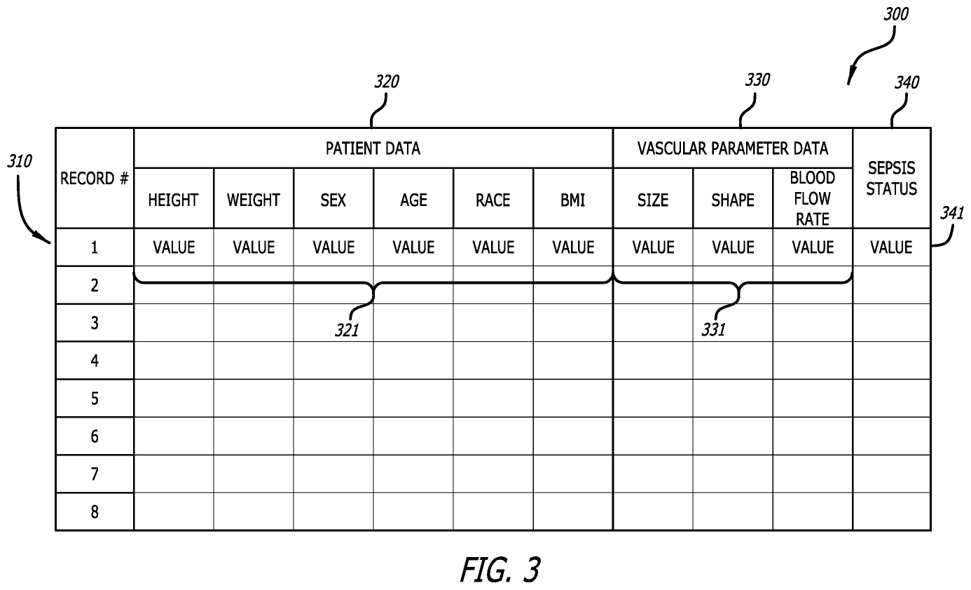

FIG. 3 illustrates an exemplary table 300 of the database 107, according to some embodiments. The table 300 includes a number (e.g., 10, 100, 1000, or more) data sets (or records) 310 as received from the plurality of sepsis monitoring systems 110 where the data set 310 may include data pertaining to a sepsis event for a patient. The data table 300 includes vascular parameter data 330, such as described above in relation FIGS. 2A-2C, for example, and sepsis status data 340. The sepsis status data 340 constitutes a sepsis status (i.e., an independently acquired sepsis status) for the patient as acquired by any suitable independent means, such as a blood test, a urine test, a patient condition, a computerized tomography (CT) scan, an X-ray, for example. The sepsis status 340 may be defined in accordance with a sepsis status ranking. For example, a ranking may include number of ranked sepsis status values, such as a minor, a medium, and a severe sepsis status, for example. As such, the sepsis status 340 may include a sepsis status value 341, e.g., one of a minor, medium, or severe sepsis status, for example. Other ranking methodologies as may be contemplated by one of ordinary skill that may also be deployed are included in this disclosure. By way of summary, each data set 310 includes at least a vascular parameter value (or set of vascular parameter values) 331 and a corresponding sepsis status value 341.

Although not required, the table 300 may also include patient data 320, according to some embodiments. Each data set 310 may include a value 321 for all or any subset of the patient data 320. The patient data 320 may include any suitable patient parameters, such as height, weight, sex, age, race, or body mass index (BMI) for example. Other patient conditions may also be included, such as blood pressure, pulse rate, medications, or body temperature, for example. The inclusion of the patient data 320 may enable the AI logic 109 to enhance an accuracy of the correlation or the algorithm. The AI logic may define the algorithm based on the data table 300 (i.e., the contents or data included in the data table 300).

The AI logic 109 may continue to collect or receive additional data sets from the plurality of sepsis monitoring systems 110 to refine the algorithm (e.g., increase a confidence level of the algorithm). In some embodiments, the AI logic 109 may regularly (e.g., continually) receive addition data sets. In some embodiments, the AI logic 109 may define/calculate of a confidence level (or more than one confidence level) for the algorithm. In such embodiments, the AI logic 109 may transmit the confidence level to the sepsis monitoring systems 110. In some embodiments, the sepsis logic 119 may provide a notification that indicates the confidence level.

The sepsis logic 119 may be generally configured to determine an instant sepsis status of the patient 50 in accordance with an instant vascular parameter value as received from the imaging logic 118. According to a first exemplary implementation, the sepsis logic 119 may (i) receive the algorithm from the AI logic 109, (ii) receive the instant vascular parameter value from the imaging logic 118, and (iii) apply the algorithm to the instant vascular parameter value to determine the instant sepsis status (e.g., determine that the instant sepsis status is minor, medium, or severe). The sepsis logic 119 may also repeatedly determine the instant sepsis status to monitor or track a progression the sepsis status during a sepsis event.

The clinician may, during the sepsis event or at the conclusion thereof, independently acquire an instant sepsis status as described above. The clinician may input the instant independent sepsis status (e.g., minor, medium, or severe) into the system module 111, such as via the display 112, for example, where the instant independent sepsis status corresponds to the instant vascular parameter value. The sepsis logic 119 may then transmit the instant vascular parameter value and the corresponding instant independent sepsis status to the external computing device 60 for inclusion in the data table 300 as a data set 310.

According to a second exemplary implementation, sepsis logic 119 may receive instant patient data values including all or any subset of the patient data 320 for the instant patient (i.e., the patient 50). The sepsis logic 119 may transmit the instant patient data values to the external computing device 60 for processing by the AI logic 109. The AI logic 109 may process the instant patient data values and determine an instant algorithm specific to the patient 50 in accordance with the data table 300. The AI logic 109 may transmit the instant algorithm to the system 110A. According to a second exemplary implementation, the sepsis logic 119 may (i) receive the instant algorithm from the AI logic 109, (ii) receive the instant vascular parameter value from the imaging logic 118, and (iii) apply the instant algorithm to the instant vascular parameter value to determine that the instant sepsis status for the patient 50 is minor, medium, or severe. The sepsis logic 119 may also repeatedly determine the instant sepsis status to monitor the sepsis status during a sepsis event.

The clinician may input the instant independent sepsis status (e.g., minor, medium, or severe) into the system module 111, such as via the display 112, for example, where the instant independent sepsis status corresponds to the instant vascular parameter value. The sepsis logic 119 may then transmit the instant vascular parameter value and the corresponding instant independent sepsis status to the external computing device 60 for inclusion in the data table 300 as a data set 310 that includes the instant patient data values. As may be contemplated by one of ordinary skill, other implementations may also be deployed that utilize historical vascular parameter data, independent sepsis status data to determine instant sepsis status based on an instant vascular parameter value.

In some embodiments, the sepsis logic 119 may compare the instant sepsis status with a number of ranked sepsis statuses (e.g., minor, medium or severe) stored in the memory 117. The sepsis logic 119 may provide a notification when the instant sepsis status exceeds a defined one of the ranked sepsis statuses. For example, the sepsis logic 119 may cause an audible alarm to sound or a visual indication to be rendered on the display 112, when the instant sepsis status exceeds the medium or severe sepsis status stored in the memory 117.

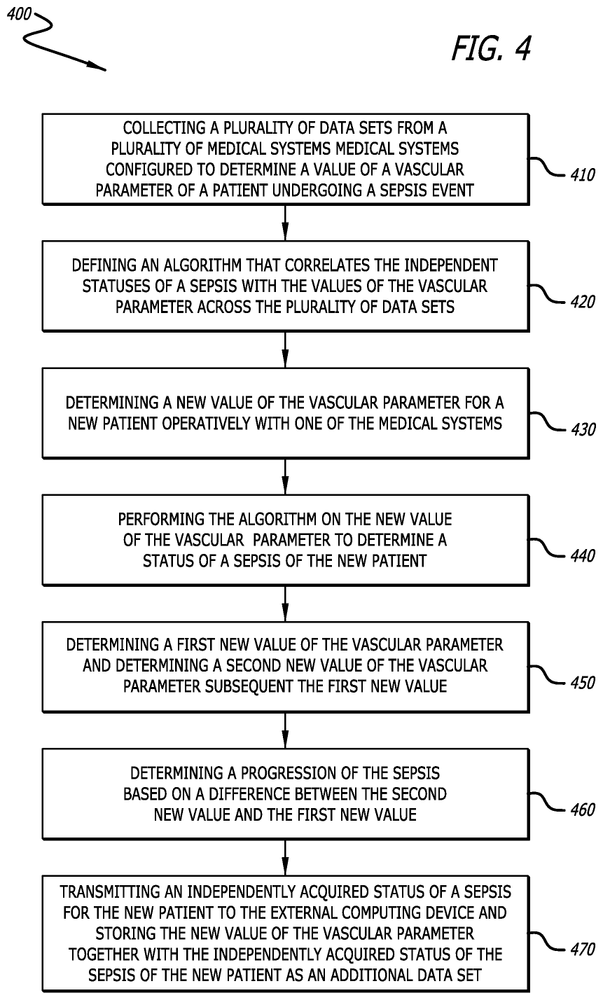

FIG. 4 is a block diagram of a system method of determining a status of a sepsis of a patient that, according to some embodiments, includes all or any subset of the following actions, steps, or processes. The method 400 may include collecting a plurality of data sets from a plurality of medical systems (block 410). Each data set includes a value of the vascular parameter and an independently acquired status of a sepsis corresponding to the value of the vascular parameter. In some embodiments of the method 400, each medical system includes a pad having a number of ultrasonic transducers extending across a patient contact surface of the pad, the ultrasonic transducers configured to acquire the value of the vascular parameter. In some embodiments of the method, the vascular parameter includes at least one of a size of a blood vessel, a shape of the blood vessel, or a blood flow rate through the blood vessel.

The method 400 may further include defining an algorithm that correlates the independent statuses of the sepsis with the values of the vascular parameter across the plurality of data sets (block 420). The method 400 may further include, determining a new value of the vascular parameter for a new patient operatively with one of the medical systems (block 430), and performing the algorithm on the new value of the vascular parameter to determine a status of a sepsis of the new patient (block 440).

In some embodiments, the method 400 may further include determining a first new value of the vascular parameter and determining a second new value of the vascular parameter (block 450), where the second new value is acquired subsequent the first new value. In such embodiments, the method 400 may further include determining a progression of the sepsis based on a difference between the second new value and the first new value (block 460).

In some embodiments of the method 400, the determining a new value of the vascular parameter includes determining a new value of the vascular parameter for a first vasculature via a first pad and determining a new value of the vascular parameter for a second vasculature via a second pad, where the second vasculature is spaced away from the vasculature.

In some embodiments of the method 400, each data set includes patient data that correspond to the vascular parameter and the independent status of the sepsis, and the patient data include one or more of weight, height, sex, age, race, or body mass index.

In some embodiments of the method 400, the one of the medical systems is communicatively coupled with an external computing device. The method 400 may further include storing the plurality of data sets in a memory of the external computing device and transmitting new values of patient data for the new patient to the external computing device. In such embodiments of the method 400, the defining of the algorithm is performed by the external computing device based on the plurality of data sets and the new values of patient data for the new patient, and the method 400 may further include receiving the algorithm from the external computing device.

In some embodiments, the method 400 may further include transmitting an independently acquired status of a sepsis for the new patient to the external computing device, and storing the new value of the vascular parameter together with the independently acquired status of the sepsis of the new patient in the database as an additional data set (block 470).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical system, comprising:
a first pad applied to a skin surface of a patient, the pad configured to acquire an image of a first vasculature of the patient,
a system module having a console coupled with the first pad, the console including a processor and a memory having logic stored thereon that, when executed by the processor performs operations that include:
receiving the image of the first vasculature from the first pad;
determining a value of a vascular parameter of the first vasculature from the image of the first vasculature;
determining a status of a sepsis of the patient based on the value of the vascular parameter of the first vasculature.

2. The system according to claim 1, wherein the first pad includes a number of ultrasonic transducers extending across a patient contact surface of the first pad, the ultrasonic transducers configured to acquire the image of the first vasculature.

3. The system according to claim 1, wherein:
the image of the first vasculature includes a first blood vessel, and
the vascular parameter includes a size of the first blood vessel, a shape of the first blood vessel, or a blood flow rate through the first blood vessel.

4. The system according to claim 1, wherein the vascular parameter is one of a plurality of vascular parameters that includes at least two of the size of the first blood vessel, the shape of the first blood vessel, or the blood flow rate through the first blood vessel.

5. The system according to claim 1, wherein the operations further include:
receiving a first image of the first vasculature from the first pad;
receiving a second image of the first vasculature from the first pad, the second image subsequent the first image;
determining a first value of the vascular parameter of the first vasculature from the first image;
determining a second value of the vascular parameter of the first vasculature from the second image;
determining a difference between the second value and the first value; and
determining a progression of the sepsis based on the difference.

6. The system according to claim 1, wherein:
the image of the first vasculature includes a second blood vessel of the first vasculature, and
the vascular parameter includes at least one of a size of the second blood vessel, a shape of the second blood vessel, or a blood flow rate through the second blood vessel.

7. The system according to claim 1, further comprising:
a second pad applied to the skin surface at a location separate from the first pad, the second pad configured to acquire an image of a second vasculature of the patient,
wherein the console is coupled with the second pad, and the operations further include:
receiving the image of the second vasculature from the second pad;
determining a value of a vascular parameter of the second vasculature from the image of the second vasculature; and
determining the status of the sepsis based on the value of the vascular parameter of the second vasculature in combination with the value of the vascular parameter of the first vasculature.

8. The system according to claim 1, wherein:
the determining a status of a sepsis includes performing an algorithm on the value of the vascular parameter, and
the algorithm is defined at least partially by historical data from a plurality of sepsis events across a population of patients.

9. The system according to claim 8, wherein the historical data include values of the vascular parameter and corresponding statuses of the sepsis.

10. The system according to claim 9, wherein:
the historical data further include corresponding patient data, and
the corresponding patient data include one or more of weight, height, sex, age, race, or body mass index.

11. The system according to claim 1, wherein the system module is communicatively coupled with an external computing device configured to:
receive the value of the vascular parameter from the system module,
generate the algorithm based on the value of the vascular parameter and the historical data, and
transmit the algorithm to the system module.

12. The system according to claim 1, wherein the operations further include:
comparing the status of the sepsis with a plurality of ranked sepsis statuses stored in the memory, and
providing a notification when the status of the sepsis exceeds a defined one of the ranked sepsis statuses.

13. A system method of determining a status of a sepsis of a patient, comprising:
collecting a plurality of data sets from a plurality of medical systems, wherein:
each medical system is configured to determine a value of a vascular parameter of a patient undergoing a sepsis event, and
each data set includes a recorded value of the vascular parameter and an independently acquired status of a sepsis corresponding to the recorded value of the vascular parameter;
defining an algorithm that correlates the independent acquired statuses of the sepsis with the recorded values of the vascular parameter across the plurality of data sets;
determining a new value of the vascular parameter for a new patient via one of the medical systems; and
performing the algorithm on the new value of the vascular parameter to determine a status of a sepsis of the new patient.

14. The method according to claim 13, wherein each medical system includes at least one pad having a number of ultrasonic transducers extending across a patient contact surface of the pad, the ultrasonic transducers configured to acquire the value of the vascular parameter.

15. The method according to claim 13, wherein the vascular parameter includes at least one of a size of a blood vessel, a shape of the blood vessel, or a blood flow rate through the blood vessel.

16. The method according to claim 13, further comprising:
determining a first new value of the vascular parameter;
determining a second new value of the vascular parameter, the second new value acquired subsequent the first new value;
determining a difference between the second new value and the first new value; and
determining a progression of the sepsis based on the difference.

17. The method according to claim 13, wherein the determining a new value of the vascular parameter includes:
determining a new value of the vascular parameter for a first vasculature via a first pad; and
determining a new value of the vascular parameter for a second vasculature via a second pad, the second vasculature spaced away from the first vasculature.

18. The method according to claim 13, wherein:
each data set includes corresponding patient data that correspond to the vascular parameter and the independently acquired status of the sepsis, and
the corresponding patient data include one or more of weight, height, sex, age, race, or body mass index.

19. The method according to claim 13, wherein the one of the medical systems is communicatively coupled with an external computing device, the method further comprising:
storing the plurality of data sets in a memory of the external computing device;
transmitting new values of corresponding patient data for the new patient to the external computing device;
defining the algorithm based on the plurality of data sets and the new values of corresponding patient data for the new patient; and
receiving by the one of the medical systems the algorithm from the external computing device.

20. The method according to claim 19, further comprising:
transmitting an independently acquired status of the sepsis of the new patient to the external computing device; and
storing the new value of the vascular parameter together with the independently acquired status of the sepsis as an additional data set.
